(19) United States
(12) Patent Application Publication
(10) Pub. No.: US 2021/0260271 A1
(43) Pub. Date: Aug. 26, 2021

(54) METHOD AND SYSTEM FOR REDUCING NOISE OF A WEARABLE MEDICAL DEVICE

(71) Applicant: KCI LICENSING, INC., San Antonio, TX (US)

(72) Inventors: Christopher B. LOCKE, Bournemouth (GB); Justin A. LONG, Bournemouth (GB)

(21) Appl. No.: 17/252,541

(22) PCT Filed: Jun. 11, 2019
(86) PCT No.: PCT/US2019/036534

§ 371 Date: Dec. 15, 2020

Related U.S. Application Data
(60) Provisional application No. 62/689,353, filed on Jun. 25, 2018.

(51) Int. Cl. A61M 1/00; A61F 13/00; A61F 13/02

(52) U.S. Cl. CPC A61M 1/962; A61M 1/80; A61F 13/00068; A61M 2209/088; A61M 1/784; A61M 2205/42; A61M 2205/8206; A61F 13/0216

(57) ABSTRACT

A reduced-noise pump system for use with a wearable medical device includes a pump housing. The pump system may be used with any number of different wearable medical devices, including wearable negative pressure wound therapy devices. Provided within the pump housing is a pump, a power source and one or more acoustically insulating elements. Examples of acoustically insulating elements that may be provided within the pump housing are an encasement layer, a baffle, and a pump cover. A control unit may be optionally also be provided, with the control unit being configured to under-drive the power source of the pump system in order to reduce the operating noise of the pump.

### CROSS-REFERENCE TO RELATED APPLICATIONS

This application claims the benefit of priority to U.S. Provisional Application No. 62/689,353, filed on Jun. 25, 2018, which is incorporated herein by reference in its entirety.

### BACKGROUND

The present disclosure relates generally to a noise reducing pump system, and more particularly, but without limitation, to a noise reducing pump system for use with a wearable medical device.

Following (or in lieu of) clinic-based medical treatments and/or medical treatments performed using a therapy device located external to a patient, patients having a medical condition are often required to continue treatment using a wearable medical device. In contrast to clinic-based treatments and/or treatments using externally located therapy devices, during treatments using a wearable medical device, the patient is typically not confined to a clinical setting and the components of the wearable medical device are incorporated into a housing unit or system that is intended to be entirely supported on or by the patient.

The arrangement of the components of a wearable medical device into a patient supported housing unit or system untethers patients from stationary treatment devices and provides patients with the freedom to carry out normal day-to-day activities outside a clinical setting. However, despite the convenience and many benefits that portable, wearable medical devices provide, patients undergoing treatment by such devices often complain about the sounds emitted by such devices during their operation. In particular, patients commonly report feeling self-conscious about the unwanted attention that is caused by and/or being distracted by the noticeable, audible noise coming from the device, with many patients admitting to removing and/or powering-off the device in such scenarios.

Given the detrimental effect that the audible operation of a wearable device may have on patient compliance with treatment, it would be desirable to provide a quiet, low-profile noise reducing pump system that would allow for the discrete and quiet use of a wearable medical device.

### SUMMARY

One implementation of the present disclosure is a wearable, self-contained, negative pressure wound therapy device that includes a dressing module and a pump module. The dressing module includes a dressing, a drape, and a dressing interface. The dressing is configured for placement in or over a wound. The drape is configured for placement over the dressing and for sealing to a patient proximate the wound. The dressing interface connector is disposed on the drape and has an opening communicating with the dressing.

The pump module is configured to be releasably connected to the dressing module. The pump module includes a top cover, a bottom cover, a pump interface, a pump, a battery and acoustic insulation. The top cover and a bottom cover define a cavity therebetween. The pump interface connector defines a suction passageway therethrough and is configured to reasonably engage the dressing interface connector. The pump is disposed within the cavity and is operably coupled to the pump interface connector to draw a suction through the suction passageway. The battery is disposed within the cavity and is coupled to the pump. The acoustic insulation is disposed within the cavity proximate the pump.

In some embodiments, the acoustic insulation includes a foam material. In some embodiments, the acoustic insulation includes a muffler operably coupled to at least one of a suction or a discharge of the pump. The muffler includes baffles formed from a plurality of interconnected chambers.

In some embodiments, the dressing interface connector and the pump interface connector are mutually engageable in a snap-fit connection. In some embodiments, a filter is disposed proximate the dressing interface connector.

One implementation of the present disclosure is a noise-reducing pump housing for a wearable therapy device that includes a top layer, a bottom layer, a pump and a noise reducing element. The top layer has an upper surface and a lower surface. The bottom layer has an upper surface and a lower surface. An outer periphery of the lower surface of the top layer is sealed to an outer periphery of the upper surface of the bottom layer to define a housing interior. The pump is located within the housing interior. The noise reducing element is attached to the pump.

In some embodiments, a noise reducing layer is located within the housing interior. The noise reducing layer includes a foam padding. The outer dimensions of the noise reducing layer correspond substantially to the dimensions of the housing interior.

In some embodiments, an opening is formed in the noise reducing layer. An outer periphery of the opening corresponds to outer peripheries defined by the pump and noise reducing element. In some embodiments, the opening extends entirely through the noise reducing layer. In some embodiments, the opening extends partially through the noise reducing layer.

In some embodiments, the top layer and bottom layer are welded together. In some embodiments, the weld may be a radio-frequency weld. One or more gaps may be formed in the weld. The gaps provide fluid communication between the housing interior and an exterior environment. The weld between the top layer and bottom layer may be waterproof.

In some embodiments, a power source is located within the housing interior. In some embodiments, a control unit is located within the housing interior. The control unit is configured to control the activation of the pump by the power source. In some embodiments, the power source includes a battery. In some embodiments, the battery is wirelessly rechargeable.

In some embodiments, the control unit is configured to control the battery to drive the pump at a voltage that is between approximately 40 percent and approximately 100 percent of the voltage capacity of the battery. In some embodiments, the battery is a 3V battery and the control unit is configured to control the battery to drive that pump at a voltage between approximately 1.5V and approximately 2.5V.

In some embodiments, an opening is formed in the bottom layer. In some embodiments, a connector element is fluidly sealed about the opening formed in the bottom layer. The connector element includes a fluid inlet and a fluid outlet. In some embodiments, an engagement element is provided on the connector element. The engagement element is configured to engage a corresponding engagement element provided on a therapy device to fluidly connect the pump housing to the therapy device.

In some embodiments, the therapy device is a wearable wound dressing. The wound dressing is attached to the pump housing via a connection between an engagement element formed on the wound dressing and the engagement element of the pump housing connector element. In some embodiments, the pump housing overlies the wound dressing. An outer perimeter of the pump housing is substantially the same as an outer perimeter of the wearable wound dressing.

In some embodiments, the pump housing and the wound dressing are constructed as an integral unit. In some embodiments, the pump housing and the wound dressing are removably detachable from one another. In some embodiments, the fluid outlet of the connector element is attached to an inlet opening of the noise reducing element.

In some embodiments, the noise reducing element includes a first plurality of interconnected chambers and a second plurality of interconnected chambers. Each of the first plurality and second plurality of interconnected chambers include an inlet chamber and an outlet chamber. Each chamber of the first plurality of chambers includes a first opening and a second opening. The first opening is linearly offset from the second opening.

In some embodiments, the first openings of the chambers of the first plurality of chambers are each arranged in a linear direction relative to one another and the second openings of the chambers of the first plurality of chambers are each arranged in a linear direction relative to one another. The arrangement of the first openings of the chambers of the first plurality of chambers is substantially parallel to the arrangement of the second openings of the chambers of the first plurality of chambers. Each chamber of the second plurality of chambers includes a first opening and a second opening. The first opening is positioned perpendicularly relative to the second opening.

In some embodiments, the inlet opening of the noise reducing element is defined by the inlet chamber of the first plurality of chambers and an outlet opening of the noise reducing element is defined by the outlet chamber of the second plurality of chambers. In some embodiments, the chambers of the second plurality of chambers are arranged such that the first openings and the second openings of the chambers of the second plurality of chambers define a generally sinuous path fluidly connecting the inlet chamber and the outlet chamber of the second plurality of chambers.

In some embodiments, the pump includes an inlet port and an outlet port. The first plurality of interconnected chambers extends between the inlet opening of the noise reducing element and the inlet port of the pump. In some embodiments, the second plurality of interconnected chambers extend between the outlet opening of the pump and an outlet opening of the noise reducing element.

In some embodiments, each of the chambers forming the noise reducing element are defined by interior surfaces formed free of any edges or corners. In some embodiments, each of the chambers forming the noise reducing element are defined by interiors having curved surfaces.

In some embodiments, the top layer and the bottom layer are each formed from a non-rigid material. In some embodiments, the top layer and the bottom layer are formed from a flexible film.

In some embodiments, the pump includes a non-piezoelectric pump. In some embodiments, the pump includes a diaphragm pump. In some embodiments, the pump includes a motor, a diaphragm, and a connecting arm extending between the motor and diaphragm. In some embodiments, the pump further includes a cover housing, the connecting arm being enclosed within the cover housing.

In some embodiments, the cover housing includes an attachment port. In some embodiments, the pump includes a vacuum port and an exhaust port, the exhaust port of the pump being fluidly attached to the attachment port of the cover housing. In some embodiments, a vent formed in the top layer. In some embodiments, the vent includes a sterile filter.

One implementation of the present disclosure is a method of replacing a negative pressure wound therapy dressing includes disconnecting a pump module from a used dressing module. The used dressing module includes a dressing, a drape disposed over the dressing, and a dressing interface connector disposed on the drape. The pump module includes a top cover and a bottom cover defining a cavity therebetween. A pump interface connector defines a suction passageway therethrough that is configured to reasonably engage the dressing interface connector. A pump is disposed within the cavity and is operably coupled to the pump interface connector to draw a suction through the suction passageway. A battery is disposed within the cavity and is coupled to the pump. Acoustic insulation is disposed within the cavity proximate the pump. A new dressing module is applied to a wound site. The pump module is connected to the new dressing module by engaging the pump interface connector to the dressing interface connector.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is top perspective view of a pump system attached to wearable medical device according to an exemplary embodiment;

FIG. 2A is an exploded top perspective view of the pump system and a top perspective view of the wearable medical device of FIG. 1 according to an exemplary embodiment;

FIG. 2B is top view of the pump system of FIG. 1 shown with the encasement layer and the top layer of the pump housing removed, according to an exemplary embodiment;

FIG. 3A is a bottom perspective view of a baffle attached to a pump, according to an exemplary embodiment;

FIG. 3B is a top perspective view of the baffle of FIG. 3A according to an exemplary embodiment;

FIG. 4A is a perspective view of a diaphragm pump having an open-crank design according to an exemplary embodiment;

FIG. 4B is a perspective view of the pump of FIG. 4A shown with an attached crank case according to an exemplary embodiment; and

FIG. 4C is a perspective view of the pump of FIG. 4A shown with an attached crank case according to an exemplary embodiment.

### DETAILED DESCRIPTION

### Overview

Many wearable medical devices are operated using a motor-driven pump. Examples of such pump-operated wearable medical devices include wearable negative pressure wound therapy (“NPWT”) systems in which the motor-driven pump of the wearable NPWT system is configured to generate a vacuum in an area surrounding a patient wound site that is to be treated and to which the wearable NPWT system is attached.

Unlike many of their clinic-based counterparts, wearable medical devices including a motor-driven pump typically are configured such that the motor-driven pump is attached to or worn by the patient during operation of the wearable medical device. Although such an arrangement of the motor-driven pump on the patient untethers the patient from the constraints of stationary clinic-based treatment devices and allows the patient to carry on with most day-to-day activities, this freedom comes as a cost.

More specifically, because the motor-driven pump of the wearable medical device is attached to or worn by the patient, this close proximity of the motor-driven pump to the patient often results in the patient being subject to the audible noises that are generated by the motor-driven pump during operation of the wearable medical device. Accordingly, despite the convenience of wearable pump-driven medical devices, the effectiveness of treatment provided by these devices is often compromised, as many patients admit to turning off and/or removing their wearable pump-driven device(s) in situations in which they feel self-conscious about and/or distracted by the noise levels emanating from the wearable device during its operation.

Generally, the noise levels of wearable pump-operated medical devices can be attributed to two main sources: mechanical/electrical noise (i.e. noise attributable to the operation of the pump) and pneumatic noise (i.e. noise attributable to the movement of air/fluid that is displaced during operation of the pump). Prior attempts to provide quieter wearable pump-operated medical devices have relied on replacing the motor-operated diaphragm pumps typically used with wearable medical devices with less-audible piezoelectric (PZT) pumps and/or on encasing the pump and motor used to power the wearable medical device in a rigid housing to muffle the sounds of these components.

Although these PZT-based and/or rigid-housing based attempts to reduce noise have shown some success, there are notable disadvantages to these solutions. In particular, while PZT pumps may operate effectively silently, and thus generate less noise than their diaphragm-based pump alternatives, when compared to diaphragm-based pumps, PZT pumps are extremely electrically and pneumatically inefficient; generate a large amount of heat during operation; consume a large amount of power; have a short life-span; and are less reliable. As a result, although substituting a diaphragm pump with a PZT pump in a wearable medical device may provide for a quieter wearable medical device, doing so requires substantial additional modifications to the wearable device, such as, e.g. incorporating a heat management system to mitigate the additional heat generated by the PZT pump, utilizing a more expensive and higher energy density battery to drive the energy-consuming PZT pump, etc.

Similarly, the option of encasing the pump within a rigid housing is also a problematic solution for reducing the level of audible noise generated by a wearable medical device. In particular, the design of most wearable medical devices is adapted to be both low-profile (so as to allow for discrete use of the wearable medical device) and comfortable to wear. Although encasing a pump and motor within a rigid-housing may help in deadening noise emanating from the wearable medical device, doing so may come at the expense of making the wearable medical device less comfortable to wear as well as bulkier and thus more difficult to conceal. As such, while encasing the pump of a wearable medical device in a rigid housing may alleviate issues related to audible noise during operation of the wearable medical device, the self-consciousness caused by the conspicuousness of the resultant rigid-housing based wearable medical device and/or the discomfort caused by wearing a rigid-housing based wearable medical device could negatively impact patient compliance with wearing and using the wearable medical device as intended.

In light of the sacrifices to comfort, discreteness, reliability, energy-efficiency, cost-savings and minimal heat generation related to the incorporation of PZT pumps and/or rigid housing in conventional wearable medical device noise-reducing pump housings, it would be desirable to provide a noise-reducing pump housing for incorporation into and/or use with a wearable medical device that would provide for equal or greater noise-reducing capabilities as compared to current noise-reducing solutions based on the use of PZT pumps and/or rigid-housings.

### Noise Reducing Pump System

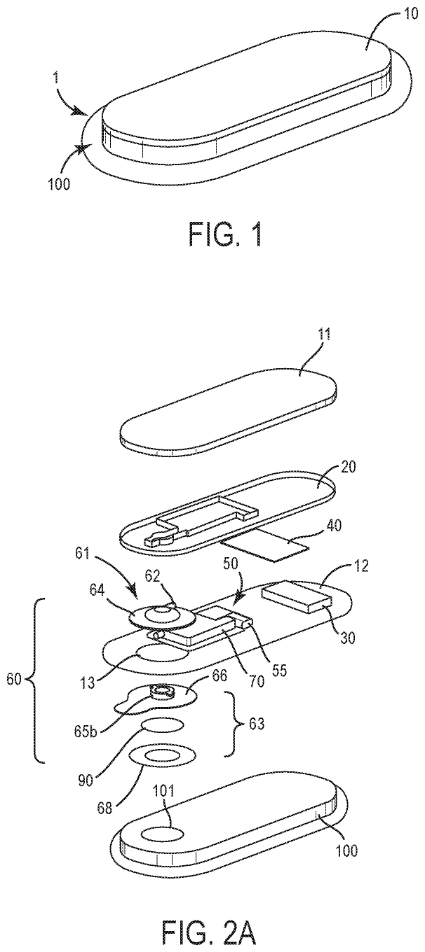

Referring to FIGS. 1-4, a low-profile, non-rigid, discrete noise-reducing pump system 1 according to various embodiments is shown. As shown in FIG. 1, in various embodiments, the noise-reducing pump system 1 may be used in conjunction with (e.g. be attached to or incorporated into) a wearable medical device 100, such as, e.g. a wearable negative pressure wound therapy (“NPWT”) device. According to various embodiments, the wearable NPWT device may comprise a PREVENA™ system as available from Kinetic Concepts, Inc. (KCI) of San Antonio, Tex.

As described in more detail below, the noise-reducing pump system 1 may include a number of elements and features that are intended to reduce the level of noise that is audible during operation of the pump 50 of the pump system 1. In general, the noise-reducing pump system 1 comprises a pump housing 10 within which a pump 50, a power source, such as e.g. a battery 30, for driving the pump 50, and an optional control unit 40 adapted to control the power source are located. The noise-reducing pump system 1 may also include a dressing interface 60 adapted to attach the noise-reducing pump system 1 to a wearable medical device 100. Noise-reducing elements adapted to reduce the audibility of the pump 50 during operation of the wearable medical device 100, such as a baffle 70, a crank case 57 and/or an encasement layer 20 may also be provided as part of the noise-reducing pump system 1.

i. Pump Housing

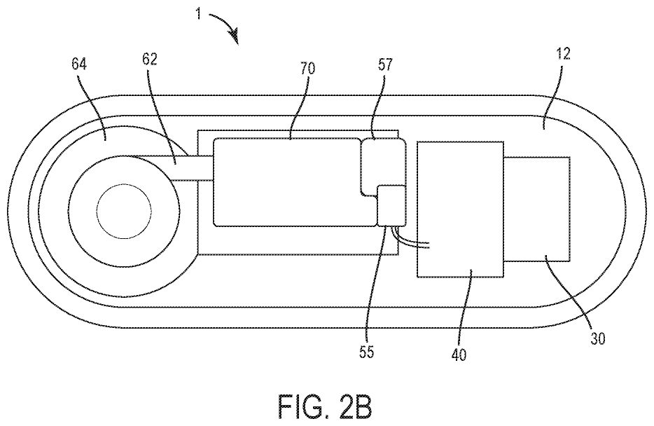

Referring to FIGS. 1, 2A and 2B, the pump housing 10 is generally formed from an upper layer 11 and a bottom layer 12 attached to one another about their respective peripheries so as to define a pump housing 10 interior. The upper layer 11 and bottom layer 12 may be attached about their outer peripheries to form pump housing 10 according to any number of know attachment arrangements, such as e.g. via a radiofrequency or other type of weld, using an adhesive, etc.

Advantageously, each of the upper layer 11 and the bottom layer 12 are formed from non-rigid, thin, flexible and elastic materials, such that the dimensions and profile of the pump housing 10 may be minimized as much as possible, allowing the pump housing 10 to be worn discretely and comfortably by a user. Non-limiting examples of materials that may be used for the upper layer 11 and/or bottom layer 12 include polymers such as, e.g., urethane or polyvinylchloride.

In various embodiments, such as, e.g. embodiments in which the pump housing 10 is used with a wearable medical device 100 comprising a NPWT system, one or more vent elements (not shown) may be provided about the pump housing 10 to vent air displaced from a wound site about which the NPWT system is placed. The vent elements are advantageously configured to allow air to be expelled from the pump housing 10 while maintaining the pump housing 10 substantially water-proof and sterile. In some embodiments, the venting element may comprise one or more weld gaps formed about the attachment between the upper layer 11 and the bottom layer 12. In other embodiments, the venting elements may alternatively or additionally comprise one or more openings (not shown) formed about the pump housing 10 that are optionally covered by a sterile filter 90 such as described below.

In some embodiments, the pump housing 10 may be configured to be positioned at a location on or about a patient that is spaced away from the location about which the wearable medical device 100 is located, with the pump housing 10 only being operatively attached to the wearable medical device 100 via an attachment element (e.g. tubing) extending between the pump 50 and the wearable medical device 100. However, in other embodiments, the pump housing 10 may be adapted to be attached to, or be formed integral with, the wearable medical device 100, such that pump housing 10 and the wearable medical device 100 are capable of being applied to and/or removed from the patient as a single unit.

Illustrated in FIG. 1 is one exemplary embodiment of a pump housing 10 that is configured to be attached to a wearable medical device 100. As shown in FIG. 1, in various embodiments the bottom layer 12 of the pump housing 10 may be attached to an upper surface of the wearable medical device 100 about a portion of or an entirety of the bottom layer 12 of the pump housing 10. As will be understood, in other embodiments, the pump housing 10 may be configured to be attached to the wearable medical device 100 along any other surface(s) of the pump housing 10 and/or wearable medical device 100.

In embodiments in which one or more surfaces of the pump housing 10 are configured to be attached to one or more surfaces of the wearable medical device 100, the attachment surface(s) of the wearable medical device 100 and/or the attachment surface(s) of the pump housing 10 may be provided with one or attachment elements (e.g. adhesive, hook and loop fasteners, etc.) adapted to maintain the pump housing 10 and wearable medical device 100 either fixedly (i.e. non-removable) or releasably attached as an integral unit. In other embodiments, the pump housing 10 and wearable medical device 100 may be formed as a single, monolithic unit.

In embodiments in which the pump system 1 comprises a dressing interface 60 configured to attach to the wearable medical device 100 (described in more detail below), the pump housing 10 may be attached to the wearable medical device 100 via only the engagement between the attachment elements 65a, 65b of the pump connector portion 61 and the device connector portion 63 of the dressing interface 60.

Although the shape, size and dimensions of the pump housing 10 are advantageously minimized as much as possible, such that the pump housing 10 is defined by a discrete low-profile, it is to be understood that the pump housing 10 may be formed having any desired shape, size and dimensions. According to various embodiments, such as, e.g. illustrated in FIG. 1, the dimensions, shape and size of the outer periphery of the pump housing 10 may be matched to the dimensions, shape and size of the wearable medical device 100 with which the pump housing 10 is adapted to be used.

ii. Dressing Interface

In embodiments in which the pump housing 10 is formed separate from the wearable medical device 100, the pump system 1 may optionally include a dressing interface 60 configured to operably connect the pump 50 located within the pump housing 10 of the pump system 1 to a wearable medical device 100.

As shown in FIG. 2, the dressing interface 60 may generally include a pump connector portion 61 and a device connector portion 63. According to various embodiments, the pump connector portion 61 includes an adaptor 62 that is adapted to fluidly connect to one or both of the inlet port 51 and outlet port 53 of the pump 50, depending on the type of wearable medical device 100 the pump system 1 is used with. For example, in embodiments in which the pump system 1 is used with a NPWT device and the pump 50 is configured to generate a vacuum that is applied to the NPWT device, the adaptor 62 of the pump connector portion 61 may be adapted to attach to the inlet port 51 of the pump 50.

The pump connector portion 61 may further include a sealing margin 64 and an attachment element 65a configured to sealingly engage a corresponding attachment element 65b of the device connector portion 63. The device connector portion 63 generally includes a sealing margin 66 surrounding an attachment element 65b that is configured to sealingly engage the corresponding attachment element 65a of the pump connector portion 61. The attachment elements 65a, 65b of the pump connector portion 61 and the device connector portion 63 may comprise any number of interengaging structures, such as, e.g. a bayonet connection.

In embodiments in which the wearable medical device 100 is, e.g., a NPWT device, the device connector portion 63 may also include a sterile filter 90 (described in more detail below) that is adapted to isolate the pump housing 10 from the wearable medical device 100 as well as an optional adhesive ring 68 configured to attach the filter 90 to a lower surface of the sealing margin 66 of the device connector portion 63.

As shown in FIG. 2A, an opening 13 may be formed in bottom layer 12 of pump housing 10 through which the attachment elements 65a, 65b of the pump connector portion 61 and the device connector portion 63 extend to operably connect the pump 50 to the wearable medical device 100. An adhesive or other sealing element may be provided along a lower surface of the sealing margin 64 of the pump connector portion 61 to attach the pump connector portion 61 to the upper surface of the bottom layer 12 of the pump housing 10 at a location surrounding the opening 13. In other embodiments, the sealing margin 64 of the pump connector portion 61 may be formed integrally with the bottom layer 12 of the pump housing 10.

Similarly, an adhesive or other sealing element provided along an upper surface of the sealing margin 66 of the device connector portion 63 may be used to attach the device connector portion 63 to the lower surface of the bottom layer 12 of the pump housing 10 at a location surrounding the opening 13.

According to various embodiments, an adhesive or other sealing element may also be optionally provided along a lower surface of the sealing margin 66 of the device connector portion 63 that may be used to attach the device connector portion 63 to the wearable medical device 100 about an opening 101 formed along a top surface of the wearable medical device 100. Alternatively, in some embodiments, the sealing margin 66 of the device connector portion 63 of the dressing interface 60 may be formed integrally with the wearable medical device 100.

As will be understood, in such embodiments where the sealing margin 66 of the device connector portion 63 is integrally formed with the wearable medical device 100 or in which the device connector portion 63 is otherwise integrally formed with and/or attached to the wearable medical device 100, the dressing interface 60 that is provided with the pump system 1 may optionally only include the pump connector portion 61.

iii. Pump

Any number of different types of pumps 50 may be incorporated into the noise-reducing pump system 1. The type of pump 50 incorporated into the noise-reducing pump system 1 may depend on any number of different variables, such as, e.g. the type of wearable medical device 100 the pump system 1 is to be used with, the type of power source that will be incorporated into the pump system 1, the degree of anticipated use of the pump 50, whether or not the pump system 1 is configured to be reusable, etc.

For example, in some embodiments, the pump 50 may include a linear shuttle pump in which an alternating current passed through a coil surrounding a central linear stator is adapted to move a stator in a linear fashion to actuate one or more diaphragms at either end of the pump. In other embodiments, the pump 50 may be based on a flat coil/magnet system in which a coil is driven with an alternating current to move a diaphragm that in turn actuates a valve.

However, in various embodiments, the pump 50 advantageously may comprise a motor-driven diaphragm pump in which the rotary motion of a motor 55 of the pump 50 is translated and imparted onto a diaphragm forming the pump 50. Such diaphragm pumps provide numerous advantages over other types of pumps, as diaphragm pumps are highly energy efficient, and thus do not require expensive, high energy battery systems. Additionally, because diaphragm pumps generate minimal heat, the pump system 1 need not integrate a heat management element, which might otherwise need to be included into the pump housing 10. Accordingly, incorporating a diaphragm-based pump as the pump 50 of pump system 1 may allow the profile of the pump housing 10 to be minimized and made less conspicuous.

Non-limiting examples of motor-driven diaphragm pumps which may be used as the pump 50 in the pump system 1 according to various embodiments are a KVP08A wobble diaphragm pump as provided by KOGE, a 1008 series pump as provided by THOMAS GARDNER DENVER, a 2002 series pump as provided by THOMAS GARDNER DENVER, etc. The size and dimensions of the pump 50 may be varied as desired. However, to minimize the profile of the pump housing 10, according to various embodiments, the thickness of the pump (as measured between the bottom layer 12 and the upper layer 11 of the pump housing 10) is preferably no greater than approximately 20 mm, and more preferably no greater than approximately 8 mm.

iv. Baffle

During operation of the pump 50, air/fluid travelling into and/or out from the pump 50 may generate pneumatic noise. The degree of audibility of this pneumatic noise may depend on the cross-sectional area and the length of the conduit(s), such as, e.g. the inlet port 51 and or outlet port 53, through which the air/fluid passes as the air/fluid travels into and/or out from the pump 50. In general, restricting flow through the conduits(s) (and thus decreasing the velocity of the air/fluid travelling through the conduit), by e.g. decreasing the cross-sectional area of the conduit(s) and/or increasing the length of the conduit(s), will result in the frequency of the noise generated by the air/fluid being higher and thereby less noticeable.

In light of the effect of restricting flow on pneumatic noise, in various embodiments the pump system 1 may include one or more noise-reducing restriction elements that are attached to the inlet port 51 and/or outlet port 53 of the pump 50 via a substantially fluid-tight fit, and which are configured to restrict the flow of air/fluid travelling into and/or out from the pump 50 to reduce the audible level of noise generated by the pump 50.

In some embodiments, the restriction element(s) may be configured to reduce the audibility of the pneumatic noise of the air/fluid flowing into and/or out from the pump 50 by blocking a portion of the opening(s) defining the inlet port 51 and/or outlet port 53 of the pump 50, and thereby decreasing the cross-sectional area of the inlet port 51 and/or outlet port 53. In other embodiments, the restriction element(s) may define an elongated conduit that is configured to extend the length of the inlet port 51 and/or outlet port 53. In yet other embodiments, the restriction element(s) may be configured to both reduce the cross-sectional area(s) and increase the length(s) of the inlet port and/or outlet port 53.

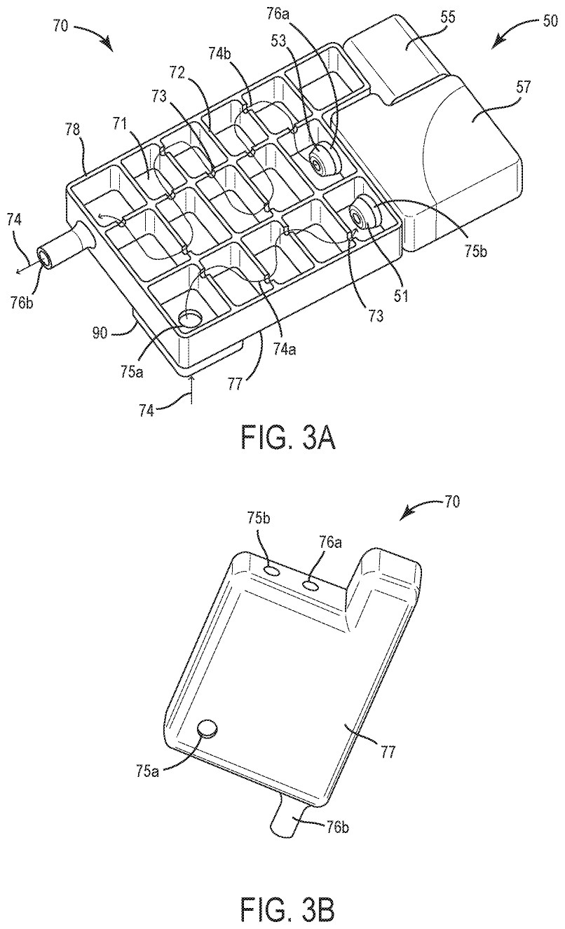

Referring to FIGS. 3A and 3B, in various embodiments, the noise-reducing restriction element may comprise a baffle 70 formed of a plurality of expansion chambers 71 that define one or more flow paths 74 configured to assist in reducing the level of noise generated during operation of the pump 50. According to various embodiments, the baffle 70 may be configured to define both an inlet flow path 74a that directs the flow of air/fluid entering into the pump 50 through the inlet port 51 as well as an outlet flow path 74b that directs the flow of air/fluid that is exhausted by the pump 50 through the outlet port 53.

As shown in FIG. 3A, in such embodiments, the inlet flow path 74a may extend between a first port 75a fluidly connected to the wearable medical device 100 (via, e.g. an attachment to the adaptor 62 of the dressing interface 60) and a second port 75b fluidly connected to the inlet port 51 of the pump 50. Meanwhile, the outlet flow path 74b may extend between a first port 76a fluidly connected to the outlet port 53 of the pump and a second port 76b. As discussed below, in some embodiments, the second port 76b of the outlet flow path 74b may optionally be fluidly attached to a port 58 on an optionally included crank case 57. In various embodiments a filter 90 may optionally be provided on one or both of the first port 75a of the inlet flow path 74a and/or the second port 76b of the outlet flow path 74b.

Although in the embodiment of baffle 70 illustrated in FIGS. 3A and 3B the baffle 70 is defined as a single, monolithic structure defining both the inlet flow path 74a and the outlet flow path 74b, in other embodiments in which the baffle 70 includes both an inlet flow path 74a and an outlet flow path 74b, the baffle 70 may include separate, non-monolithically formed structures defining each of the inlet flow path 74a and the outlet flow path 74b. Additionally, it is to be understood that in yet other embodiments, the baffle 70 may be formed to only include only one of an inlet flow path 74a or an outlet flow path 74b.

As illustrated in FIG. 3A, the tortuous flow path 74 through the baffle 70 may be defined by a series of openings 73 extending through the walls 72 defining a plurality of interconnected chambers 71. The arrangement of the chambers 71 and openings 73 may be selected such that as air flow is directed along the elongated flow path 74, the series of expansive adjacent chambers 71 and narrow openings 73 serve to cause an alternating expansion and restriction of the airflow, with the velocity—and thereby the audibility—of the airflow being reduced with each opening 73 and chamber 71 that the airflow passes through. According to various embodiments, the degree by which the noise of the airflow is reduced may further be increased by advantageously defining the walls 72 of each chamber 71 as curved surfaces formed with no or minimal flat corners or edges, such that sound may be propagated more easily as air/fluid travels between adjacent chambers 71.

In various embodiments, a further restriction on airflow through the baffle 70 may be achieved by offsetting the spacing and alignment of the openings 73 between adjacent chambers 71. As illustrated in FIG. 3A, in embodiments in which the flow path 74 extend through a series of linearly arranged chambers 71 (i.e. chambers 71 arranged along a single column, such as, e.g. the arrangement of the chambers 71 defining the inlet flow path 74a of the embodiment of baffle 70 of FIGS. 3A and 3B,) the openings 73 may be arranged such that, although each of the openings 73 are arranged in the same linear direction, the inlet opening 73a and outlet opening 73b of each chamber 71 are not aligned.

Referring to FIG. 3A, in embodiments in which the flow path 74 through the baffle 70 extends through a series of linearly offset chambers 71 (e.g. chambers 71 that are arranged in more than one column, such as, e.g. the chambers 71 defining the outlet flow path 74b of the embodiment of baffle 70 of FIGS. 3A and 3B), the inlet opening 73a and outlet opening 73b of each chamber 71 may be arranged perpendicular to one another, so as to define a sinuous flow path 74 through the baffle 70.

As illustrated in FIGS. 3A and 3B, according to various embodiments, the baffle 70 may comprise nine rounded chambers 71 that define a tortuous flow path 74 having a cross-sectional area within a range of between approximately 0.50 mm² and approximately 2.50 mm², more preferably between approximately 1.00 mm² and approximately 1.50 mm², and more preferably approximately 1.25 mm². However, as will be understood, in other embodiments, the baffle 70 may be formed having any other number of chambers 71 of any desired dimensions. As will also be understood, although the baffle 70 has been described as being defined by a structure formed of a plurality of interconnected chambers 71, in various embodiments, the baffle 70 may be formed according to any another configurations that are adapted to route airflow in such a manner as to achieve a restriction of airflow velocity, and thereby reduce noise.

The baffle 70 may be made of any desired materials. According to some embodiments, the baffle 70 may be made from a material that has sound suppressing properties, e.g. a compression molded porous mass, such as, e.g., a porous polymer and/or sintered polymer. In some embodiments, the baffle 70 may optionally additionally or alternatively include a sound suppressing liner (not shown) lining all of or a portion of the walls 72 of the baffle 70.

Referring to FIGS. 3A and 3B, in some embodiments, the baffle may be formed having a main body having a closed top surface 77 and an open bottom surface 78. As shown in FIG. 2A, in some embodiments, a fluid-tight connection between adjacent chambers 71 of the baffle 70 may be provided by a fluid tight attachment of the walls 72 defining the chambers 71 of the baffle 70 with the upper surface of the bottom layer 12 of the pump housing 10 and/or with the upper surface of the encasement layer 20 or other noise suppressing material (e.g. a soft urethane baffling layer) over which the main body of the baffle 70 is arranged. In other embodiments, the baffle may be formed having a main body having an open top surface 77 and an closed bottom surface 78, with the fluid-tight connection between adjacent chambers 71 of the baffle 70 being provided by a fluid tight attachment of the walls 72 defining the chambers 71 of the baffle 70 with the lower surface of the top layer 11 of the pump housing 10 and/or with the lower surface of the encasement layer 20 or other noise suppressing material (e.g. a soft urethane baffling layer) arranged over the main body of the baffle 70.

In other embodiments, the baffle 70 may instead be provided with a cover (not shown) that may be attached about the open surface of the main body to define a discrete closed-structure baffle 70 in which, with the exception of the fluid connection between chambers 71 defined by flow-path 74, adjacent chambers 71 of the baffle 70 are fluidly separated from one another. In yet other embodiments, the baffle 70 may be formed as a monolithic, discrete closed structure.

According to various embodiments, the pump system 1 may comprise a plurality of identical or differing baffles 70 that are arranged in series so as to further extend the length of the flow path 74 between the inlet port 51 and the outlet port 53 of the pump 50. Given that wearable medical device 100 and/or pump system 1 embodiments may not be large enough to accommodate such a series of baffles 70, it is to be understood that the incorporation of a series of baffles 70 may in some embodiments instead be used in a pump system adapted for use with a non-wearable medical device.

v. Filter

As discussed herein, according to various embodiments, one or more filters 90 may be incorporated into the pump system 1. In addition to serving to restrict flow and acting as a barrier against water and contaminants to maintain the sterility of the pump system 1, the one or more filters 90 incorporated into the pump system 1 may also act as a muffle that may assist in reducing the level of noise emanating from the pump system 1.

The filters 90 may be formed from any number of desired materials, such as, e.g. a sintered polymeric material, such as, e.g. polytetrafluoroethylene. One non-limiting example of a filter that may be incorporated into the pump system 1 is a GORE® MMT-314 membrane.

vi. Crank Case

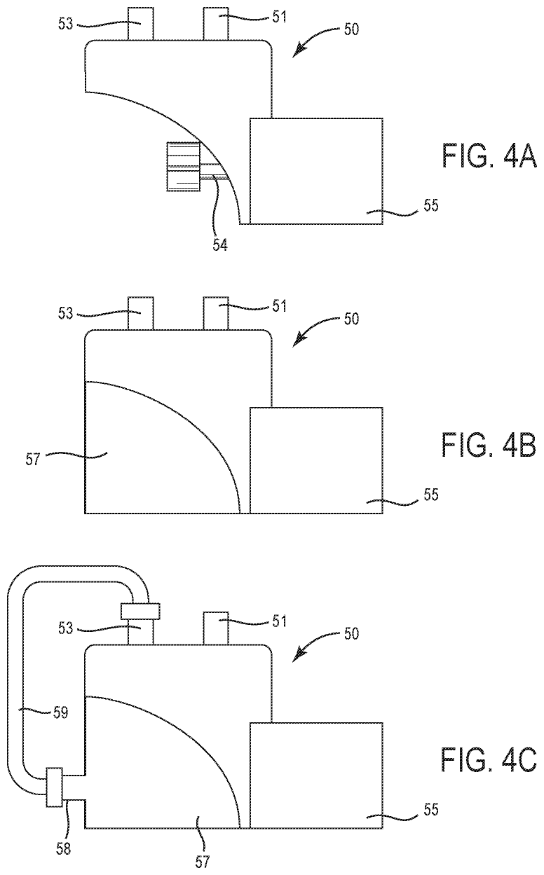

As illustrated in FIG. 4A, in various embodiments of pump system 1, the pump 50 incorporated into the pump housing 10 may be a 2002-series diaphragm-based pump as provided by THOMAS GARDNER DENVER having an open-crank design, in which an armature or crank 54 that connects the motor 55 of the pump 50 to the diaphragm (not shown) is exposed. In such embodiments, this lack of an enclosure for the crank 54 may result in the mechanical sounds emitted by the motor 55, crank 54 and/or diaphragm contributing to the overall noise generated during operation of the wearable medical device 100. Accordingly, as shown in FIGS. 4B and 4C in some embodiments, a crank case 57 that encloses the crank 54 may optionally be provided to suppress the mechanical noise generated by the operation of the pump 50.

As illustrated by the embodiment of FIG. 4B, in some embodiments, the level of noise reduction achieved simply by encasing the crank 54 within crank case 57 may be sufficient to achieve a desired decrease of the audibility of the wearable medical device 100 during its operation. However, as illustrated by the embodiment of FIG. 4C, in other embodiments, in addition to suppressing the mechanical noise emitted by the components of the motor-driven pump 50, it may be desirable to have the crank case 57 play a more active role in decreasing the audibility of the wearable medical device 100 during its operation.

More specifically, as shown in FIG. 4C, in some embodiments, a port 58 may be provided on the crank case 57. After the crank case 57 is pneumatically sealed to the pump 50, the exhaust port of the pump 50 (i.e. outlet port 53 in embodiments in which the pump 50 is adapted to generate a vacuum, such as, e.g. in embodiments in which the pump system 1 is used with a wearable medical device 100 comprising a NPWT system) is fluidly attached to the port 58, via, e.g. tubing 59. Accordingly, as air is evacuated from the area surrounding the crank 54 (i.e. the interior of the crank case 57) to generate a vacuum that is to be delivered to the wearable medical device 100 via the inlet port 51, the positive pressure exhaust from the outlet port 53 is re-routed into the interior of the crank case 57 in order to provide a white noise to mask the mechanical noise that is generated by the flow of air while the motor 55 of the pump 50 is in operation.

Although not shown, it is to be understood that in various embodiments, the pump system 1 may be configured to include both a baffle 70 and a crank case 57 formed with or without a port 58. In embodiments in which a crank case 57 having a port 58 is used in conjunction with a baffle 70, it is to be understood that exhaust from the outlet port 53 may be directed into the interior of the crank case 57 via tubing 59 that extends between the second port 76b of the outlet flow path 74b of the baffle 70 and the port 58 of the crank case 57.

vii. Power Source

Any number of different power sources may be incorporated into the pump system 1 to provide power to the pump 50. In various embodiments, the power source may include a battery 30, with the type of battery 30 that is selected being based on any number of factors, such as, e.g. the type of pump 50, whether the pump system 1 is intended to be reusable, etc. For example, embodiments of pump system 1 in which the pump 50 is a diaphragm pump may incorporate a lower voltage battery 30 than would be required if the pump 50 were a more high-energy pump 50, such as, e.g. a PZT pump.

In some embodiments, e.g. embodiments in which the pump system 1 is used with a wearable medical device 100 that is intended to be used for an extended period of time, the battery 30 may be a rechargeable battery, such that the battery 30 and/or the pump system 1 need not be replaced during treatment. In some such embodiments, the rechargeable battery 30 may advantageously be adapted to be re-charged wirelessly while in-situ (e.g. via inductive charging using a coil integrated into the pump housing 10) such that a user may recharge the battery 30 without having to remove the battery 30 from the pump housing 10 and/or without requiring exposed electrical charging connections to extend through the pump housing 10.

viii. Control Unit

As shown in FIGS. 2A and 2B, in various embodiments, the pump housing 10 may incorporate a control unit 40 configured to control the operation of the pump 50 by controlling the power delivered by the power source to drive the pump 50. As will be understood, the control unit 40 may be adapted to control the operation of the pump 50 based on any number of variables, such as, e.g. predetermined time intervals; sensed conditions at the treatment site; externally input parameters, etc.

In various embodiments, the control unit 40 may be adapted to underdrive the power source in order to reduce the level of noise emitted by the pump system 1 during operation of a wearable medical device 100 to which the pump housing 10 is attached. For example, in embodiments in which the power source comprises a battery 30, the control unit 40 may be adapted to control the battery 30 to drive the pump 50 using less than all of the voltage capacity of the battery 30.

In some such embodiments, the control unit 40 may be configured to control the battery 30 to drive the pump 50 using a voltage that is within a range of approximately 40 percent and approximately 90 percent, and more specifically within a range of approximately 70 percent and approximately 85 percent of the capacity of the battery 30. For example, in some embodiments the battery 30 may be a 3V battery, with the control unit 40 being configured to drive the battery 30 at approximately 2.5V. In another embodiment, the battery 30 may be a 2.5V battery, with the control unit 40 being configured to drive the battery 30 at approximately 1.5V. In such embodiments, the decreased level of voltage used to drive the pump 50 is still sufficient to generate the desired level of activation of the pump 50. However, because the pump 50 is driven at less than full capacity, the degree of noise generated by the pump 50 is minimized as compared to the level of noise that would otherwise be generated if the pump 50 were to be operated using the full capacity of the power source.

ix. Encasement Layer

In various embodiments, the noise-reducing pump system 1 may optionally incorporate an encasement layer 20 formed of one or more layers of sound deadening material(s) that are adapted to further reduce the audible noise levels of the pump 50. Examples of materials that may be used for the encasement layer 20 include, e.g. injection molded polymers such as silicone or SANTOPRENE™, foamed polymers, etc.

The encasement layer 20 may be formed from a single, monolithic piece of material, or the encasement layer 20 may be formed from a plurality of integrally connected or discretely arranged pieces. In addition to muffling noise that is emitted by the components of the pump system 1, the encasement layer 20 may act to dampen vibrations or other movements of the components of the pump system 1, and in doing so may assist in decreasing the mechanical noise generated by the components during operation of the pump 50.

As illustrated in FIGS. 2A and 2B, according to various embodiments, the encasement layer 20 may be adapted to generally correspond in shape and size to the dimensions of the interior of the pump housing 10 such that the encasement layer 20 surrounds each of the components located within the interior of the pump housing 10. In such embodiments, in addition to providing a noise-reducing effect, the incorporation of an encasement layer 20 into the pump system 1 may also advantageously protect the pump system 1 by acting as a barrier that protects the components of the pump system 1 from damage and/or providing a cushion that protects the patient from the rigid components of the pump system 1. In other embodiments, the encasement layer 20 may be formed to only surround a portion of the components of the pump system 1, such as, e.g. the only the pump 50.

### Method of Use

Referring to FIG. 1, according to various embodiments, the wearable medical device 100 with which the pump system 1 is used may be a NPWT device. One embodiment of a method by which the degree of noise generated by the NPWT wearable medical device 100/pump system 1 may be reduced begins with the step of forming a fluid-tight seal between the NPWT wearable medical device 100 and a patient's skin surrounding the tissue site that is to be treated. By ensuring that the seal between the patient's skin and the NPWT wearable medical device 100 is as fluid-tight as possible, the duration that the vacuum generated by the pump system 1 that is applied to the space defined between the wearable medical device 100 and the patient's skin can be maintained in maximized, thereby minimizing the need to operate the pump system 1 to maintain the vacuum at the tissue site. As noise is not generated during periods of time when the pump system 1 is not in operation, the step of providing a substantially fluid-tight seal can significantly decrease the level of noise emanating from the wearable medical device 100/pump system 1. According to various embodiments, this fluid-tight seal may be provided by using a DERMATAC™ drape as available from Kinetic Concepts, Inc. (KCI) of San Antonio, Tex.

As a second step of the exemplary method, the noise levels of the wearable medical device 100/pump system 1 may further be reduced by using the control unit 40 to under-drive the battery 30 used to power the pump 50. As described above, by driving the battery 30 within a range of approximately 40 percent and approximately 90 percent, and more specifically within a range of approximately 70 percent and approximately 85 percent, sufficient power is provided to the pump 50 to generate the desired vacuum at the tissue site, while lowering the amount of noise that would otherwise be generated had the pump 50 been driven by the full capacity of the battery 30.

Finally, the noise of the wearable medical device 100/pump system 1 may be reduced by incorporating the various noise-reducing features (e.g. crank case 57, encasement layer 20, baffle 70, etc.) as described above.

Although the noise-reducing pump system 1 has been described as being adapted for use with a wearable medical device 100, it is to be understood that a pump system 1 as described and shown herein may be also be used with any number of non-wearable medical devices or even other non-medical devices. Also, while in various embodiments the pump system 1 is disclosed as being incorporating a pump housing 10 that may be detachably or integrally attached to a wearable medical device 100, it is to be understood that in other embodiments, the various noise reducing features of the pump system 1 (e.g. baffle 70, encasement layer 20, crank case 57, etc.) may be incorporated into a housing of the wearable medical device 100 to form a single piece wearable medical device/pump system 1.

According to various embodiments, the components of the pump system 1 (e.g. the pump 50, control unit 70, power source, etc.) may be configured as being either single or multi-use. Additionally, the pump system 1 itself may be configured to be either single use or reusable. For example, in some embodiments in which the pump system 1 is used with a wearable medical device 100 comprising a NPWT system intended to be changed one or more times during a treatment period (e.g. a two-week period), the pump system 1 may be configured to be reattached to newly applied NPWT wound dressings during the treatment period, with the pump system 1 being configured to be disposed of once the treatment period has ended. In other embodiments, the pump system 1 may be adapted to be reusable, such that the pump system 1 may be reused during a subsequent treatment period to treat either the same or a different patient.

Additionally, as discussed above, in embodiments in which the pump system 1 is used with a wearable medical device 100 comprising a NPWT system, additional noise reduction may be accomplished by using a high efficiency seal that provides a long wear, such as, e.g. the use of a hydrocolloid based drape, the use of a silicone layer with the drape, or a drape incorporating DERMATAC™ sealing technology available from Kinetic Concepts, Inc. (KCI) of San Antonio, Tex., to attach the NPWT system to the patient. More specifically, by being adapted to provide a highly fluid-tight seal with the patient's skin, the ability of the drape to maintain a vacuum is increased, thus decreasing the amount by which the pump needs to be operated. By minimizing or eliminating the amount of time the pump is required to operate, the noise can be further reduced, as the pump 50 is silent when not in use.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

1. A wearable, self-contained, negative pressure wound therapy device, comprising:
a dressing module comprising:
a dressing configured for placement in or over a wound;
a drape configured for placement over the dressing and for sealing to a patient proximate the wound;
a dressing interface connector disposed on the drape and having an opening communicating with the dressing;
a pump module configured to be releasably connected to the dressing module, the pump module comprising:
a top cover and a bottom cover defining a cavity therebetween;
a pump interface connector defining a suction passageway therethrough and configured to reasonably engage the dressing interface connector;
a pump disposed within the cavity and operably coupled to the pump interface connector to draw a suction through the suction passageway;
a battery disposed within the cavity and coupled to the pump; and
acoustic insulation disposed within the cavity proximate the pump.

2. The wearable, self-contained, negative pressure, wound therapy device of claim 1, wherein the acoustic insulation comprises a foam material.

3. The wearable, self-contained, negative pressure, wound therapy device of claim 1, wherein the acoustic insulation comprises a muffler operably coupled to at least one of a suction or a discharge of the pump.

4. The wearable, self-contained, negative pressure, wound therapy device of claim 3, wherein the muffler comprises baffles formed from a plurality of interconnected chambers.

5. The wearable, self-contained, negative pressure, wound therapy device of claim 1, wherein the dressing interface connector and the pump interface connector are mutually engageable in a snap-fit connection.

6. The wearable, self-contained, negative pressure, wound therapy device of claim 1, further comprising a filter disposed proximate the dressing interface connector.

7. A noise-reducing pump housing for a wearable therapy device, the housing comprising:
a top layer having an upper surface and a lower surface;
a bottom layer having an upper surface and a lower surface, an outer periphery of the lower surface of the top layer being sealed to an outer periphery of the upper surface of the bottom layer to define a housing interior;
a pump located within the housing interior; and
a noise reducing element attached to the pump.

8. The pump housing of claim 7, further comprising a noise reducing layer located within the housing interior.

9-17. (canceled)

18. The pump housing of claim 7, further comprising a power source located within the housing interior.

19. The pump housing of claim 18, further comprising a control unit located within the housing interior.

20. The pump housing of claim 19, wherein the control unit is configured to control the activation of the pump by the power source.

21. The pump housing of claim 20, wherein the power source comprises a battery.

22. The pump housing of claim 21, wherein the battery is wirelessly rechargeable.

23-24. (canceled)

25. The pump housing of claim 7, further comprising an opening formed in the bottom layer.

26. The pump housing of claim 25, further comprising a connector element fluidly sealed about the opening formed in the bottom layer, the connector element comprising a fluid inlet and a fluid outlet.

27. The pump housing of claim 26, further comprising an engagement element provided on the connector element, the engagement element configured to engage a corresponding engagement element provided on a therapy device to fluidly connect the pump housing to the therapy device.

28. The pump housing of claim 26, wherein the therapy device is a wearable wound dressing, the wound dressing being attached to the pump housing via a connection between an engagement element formed on the wound dressing and the engagement element of the pump housing connector element.

29. The pump housing of claim 28, wherein the pump housing overlies the wound dressing, an outer perimeter of the pump housing being substantially the same as an outer perimeter of the wearable wound dressing.

30. The pump housing of claim 29, wherein pump housing and the wound dressing are constructed as an integral unit.

31. The pump housing of claim 29, wherein pump housing and the wound dressing are removably detachable from one another.

32. The pump housing of claim 26, wherein the fluid outlet of the connector element is attached to an inlet opening of the noise reducing element.

33. The pump housing of claim 32, wherein in the noise reducing element comprises a first plurality of interconnected chambers and a second plurality of interconnected chambers, each of the first plurality and a second plurality of interconnected chambers including an inlet chamber and an outlet chamber.

34-45. (canceled)

46. The pump housing of claim 7, wherein the pump comprises a non-piezoelectric pump.

47. The pump housing of claim 7, wherein the pump comprises a diaphragm pump.

48. The pump housing of claim 47, wherein the pump comprises a motor, a diaphragm, and a connecting arm extending between the motor and diaphragm.

49-58. (canceled)

59. A method of replacing a negative pressure wound therapy dressing, comprising:
disconnecting a pump module from a used dressing module, wherein the used dressing module comprising a dressing, a drape disposed over the dressing, and a dressing interface connector disposed on the drape, and wherein the pump module comprises a top cover and a bottom cover defining a cavity therebetween, a pump interface connector defining a suction passageway therethrough and configured to reasonably engage the dressing interface connector, a pump disposed within the cavity and operably coupled to the pump interface connector to draw a suction through the suction passageway, a battery disposed within the cavity and coupled to the pump, and acoustic insulation disposed within the cavity proximate the pump;
applying a new dressing module to a wound site; and
connecting the pump module to the new dressing module by engaging the pump interface connector to the dressing interface connector.
